(19) United States
(12) Patent Application Publication
(10) Pub. No.: US 2026/0176221 A1
(43) Pub. Date: Jun. 25, 2026

(54) AZEOTROPE AND AZEOTROPE-LIKE COMPOSITIONS OF 1,1,2-TRIFLUOROETHANE (HFC-143) AND HYDROGEN FLUORIDE (HF) AND APPLICATIONS THEREOF

(71) Applicant: Solstice Advanced Materials US, Inc., Morris Plains, NJ (US)

(72) Inventors: Hang T. Pham, Amherst, NY (US); Joshua Close, Cheektowaga, NY (US); Haiyou Wang, Amherst, NY (US); Terris Yang, East Amherst, NY (US)

(21) Appl. No.: 19/417,738

(22) Filed: Dec. 12, 2025

Related U.S. Application Data
(60) Provisional application No. 63/738,292, filed on Dec. 23, 2024.
(60) Provisional application No. 63/777,131, filed on Mar. 25, 2025.

(51) Int. Cl. C07C 17/087; C07C 17/38

(52) U.S. Cl. CPC C07C 17/087; C07C 17/38

(57) ABSTRACT

An azeotrope or azeotrope-like composition consisting essentially of effective amounts of 1,1,2-trifluoroethane (HFC-143) and hydrogen fluoride (HF). Methods for separating the azeotrope or azeotrope-like composition and/or exploiting the composition are also disclosed in connection with methods of manufacturing 1,1,2-trifluoroethane (HFC-143).

### CROSS-REFERENCE TO RELATED APPLICATIONS

This application claims priority to U.S. Patent Application No. 63/738,292 entitled “AZEOTROPE AND AZEOTROPE-LIKE COMPOSITIONS OF 1,1,2-TRIFLUOROETHANE (HFC-143) AND HYDROGEN FLUORIDE (HF) AND APPLICATIONS THEREOF” filed on Dec. 23, 2024, and U.S. Provisional Patent Application No. 63/777,131 entitled “AZEOTROPE AND AZEOTROPE-LIKE COMPOSITIONS OF 1,1,2-TRIFLUOROETHANE (HFC-143) AND HYDROGEN FLUORIDE (HF) AND APPLICATIONS THEREOF,” filed on Mar. 25, 2025, the entire disclosures of which are incorporated by reference in their entireties.

### FIELD

The present disclosure relates to azeotrope and azeotrope-like compositions and, in particular, to azeotrope and azeotrope-like compositions consisting essentially of 1,1,2-trifluoroethane (HFC-143) and hydrogen fluoride (HF) and applications or uses for these compositions.

### BACKGROUND

Fluorocarbon fluids have properties that are desirable for use as heat transfer media, immersion coolants, liquid or gaseous dielectrics, industrial refrigerants, and other applications.

For example, 1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents. Improved methods for the production of HFO-1132 and, in particular, HFO-1132E, are desired.

An “azeotrope” composition is a unique combination of two or more components for which compositions of vapor and liquids are the same which yield and minimum or maximum in saturation temperature or pressure. As an extension, azeotrope can be classified as being homogeneous or heterogeneous. According to Seader and Henley (Separation Process Principles, Wiley, Second Edition, 2006, pp. 123-126), if only one liquid phase exists, the mixture forms a homogeneous azeotrope; if more than one liquid is present, the azeotrope is heterogeneous. For a fixed temperature, heterogeneous azeotropes, according to Seader and Henley, have total pressures and phase compositions that remain constant across the multiphase region (a region that is sometimes referred to as the “miscibility gap”). In contrast, for a fixed temperature, homogeneous azeotropes yield only one unique total pressure where phase compositions are constant given that a multiphase region is, by definition, absent. While both heterogeneous and homogeneous azeotropes share features of constant composition, the presence or absence of a liquid multiphase region yields a difference that permits or prevents their application, use, and/or otherwise treatment of the resulting azeotrope; therefore, distinguishing an azeotrope as homogeneous or heterogeneous becomes critically important and necessary for their application, use, and/or otherwise treatment.

In U.S. Pat. No. 11,826,596 B2, Example 1, Daikin Industries, LTD. disclose the formation of a heterogeneous azeotrope between 1,1,2-trifluoroethane (HFC-143) and hydrogen fluoride (HF). Although the experimental methods required for observing azeotropes are not described, Table 1 of Example 1 in 11,826,596 B2 reveals details that are consistent with a heterogeneous azeotrope (or “heterophase” azeotrope as described within the art); total pressure remains constant (0.539 MPa) at a fixed temperature (40° C.) for a range of overall liquid compositions in a multiphase region (20 to 95 mol % of HFC-143). In contrast, as shown herein, phase behavior observed from experimental measurements for binaries of HFC-143 and HF are most consistent with a minimum boiling, homogeneous azeotrope.

Azeotrope and azeotrope-like compositions may be encountered during the manufacture of fluorocarbon fluids and understanding any such azeotrope or azeotrope-like compositions is helpful to improve the efficiency of the manufacturing processes.

### SUMMARY

The present disclosure provides minimum-boiling, homogenous azeotrope or azeotrope-like compositions consisting essentially of 1,1,2-trifluoroethane (HFC-143) and hydrogen fluoride (HF), and applications or uses for these compositions.

In one form thereof, the present disclosure provides an azeotrope or azeotrope-like composition consisting essentially of effective amounts of 1,1,2-trifluoroethane (HFC-143) and hydrogen fluoride (HF).

In another form thereof, the present disclosure provides a method for producing 1,1,2-trifluoroethane (HFC-143) comprising: hydrogenating an ethylene-based precursor molecule of the formula R₁R₂C=CR₃R₄, wherein R₁, R₂, R₃, and R₄ are selected from H, Cl, F, Br and I, with hydrogen fluoride (HF) to form a product mixture, the product mixture comprising a homogenous azeotrope or azeotrope-like composition consisting essentially of effective amounts of 1,1,2-trifluoroethane (HFC-143) and hydrogen fluoride (HF); and separating the 1,1,2-trifluoroethane (HFC-143) and hydrogen fluoride (HF) to provide a product composition comprising the 1,1,2-trifluoroethane (HFC-143).

### BRIEF DESCRIPTION OF THE DRAWINGS

FIG. 1 is a process flow diagram of a process for separating 1,1,2-trifluoroethane (HFC-143) and hydrogen fluoride (HF).

FIG. 2 is a graph of PTx measurements for an azeotrope or azeotrope-like composition of 1,1,2-trifluoroethane (HFC-143) and hydrogen fluoride (HF) at an average temperature of 49.9° C.

FIG. 3 is a schematic of an apparatus for the separation by water absorption/extractive distillation of an azeotrope or azeotrope-like composition of 1,1,2-trifluoroethane (HFC-143) and hydrogen fluoride (HF).

FIG. 4 is a schematic of an apparatus for the separation by sulfuric acid absorption/extractive distillation of an azeotrope or azeotrope-like composition of 1,1,2-trifluoroethane (HFC-143) and hydrogen fluoride (HF).

### DETAILED DESCRIPTION

The present disclosure provides minimum-boiling, homogenous azeotropic or azeotrope-like compositions consisting essentially of 1,1,2-trifluoroethane (HFC-143) and hydrogen fluoride (HF) and applications or uses for these compositions.

### I. Description of Azeotrope or Azeotrope-Like Compositions

An “azeotrope” composition is a unique combination of two or more components. An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). At this characteristic temperature the same composition will exist in both the vapor and liquid phases. The azeotrope composition does not fractionate upon boiling or evaporation. Therefore, the components of the azeotrope composition cannot be separated during a phase change.

An azeotrope composition is also characterized in that, at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

Azeotrope or azeotrope-like compositions can be identified using a number of different methods.

Static Vapor-Liquid Equilibrium Methods are a class of experimental techniques that can also be used to identify the presence of azeotrope and azeotrope-like compositions. One such technique, known as the PTx method, collects measurements of the total saturation pressure (“P”) exerted by mixtures of known compositions (“x”) at fixed temperatures (“T”) and cell volumes. (Walas, Phase Equilibria in Chemical Engineering, Butterworth-Heinemann, 1985, pp. 537). Using data collected from the PTx experiment, as well as pure component properties of constituents of the mixtures, the thermodynamic properties of the mixture can be accurately characterized by fitting the component's interaction parameters in a well-defined thermodynamic equation; one such equation is the Non-random, Two-Liquid (NRTL) activity coefficient model described by Renon and Prausnitz (Local Compositions in Thermodynamic Excess Functions for Liquid Mixtures, AIChE Journal, Vol. 14, January 1968, pp. 135-144).

The presence of an azeotrope and its corresponding composition can be observed by plotting saturation pressure measurements from PTx data and saturation pressures described by NRTL as a function of composition. For a given temperature (isotherm), the presence of an azeotrope composition is identified by the observation of a maximum or minimum in total pressure that is greater or less than the pure saturation pressures of any of the components alone.

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

As used herein, the term “about”, when used in connection with recited weight percentages of the components of the present compositions, includes a deviation of ±0.3% from the recited weight percentage.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As previously discussed, for an azeotrope, at the maximum or minimum boiling point, the composition of the vapor phase will be identical to the composition of the liquid phase. The azeotrope-like composition is therefore that composition of components which provides a substantially constant minimum or maximum boiling point at which substantially constant boiling point the composition of the vapor phase will be substantially identical to the composition of the liquid phase.

### II. Azeotrope and Azeotrope-Like Compositions of 1,1,2-Trifluoroethane (HFC-143) and Hydrogen Fluoride (HF)

The present disclosure provides a minimum-boiling, homogenous azeotrope or azeotrope-like composition comprising effective amounts of 1,1,2-trifluoroethane (HFC-143) and hydrogen fluoride (HF). The present disclosure particularly provides a minimum-boiling, homogenous azeotrope or azeotrope-like composition consisting essentially of effective amounts of 1,1,2-trifluoroethane (HFC-143) and hydrogen fluoride (HF). The present disclosure provides a minimum-boiling, homogenous azeotrope or azeotrope-like composition consisting of effective amounts of 1,1,2-trifluoroethane (HFC-143) and hydrogen fluoride (HF).

The homogenous azeotrope or azeotrope-like composition may comprise from about 88.3 wt. % to about 90.6 wt. % of 1,1,2-trifluoroethane (HFC-143) and from about 11.7 wt. % to about 9.4 wt. % of hydrogen fluoride (HF) at a temperature of from about −0.2° C. to about 49.9° C. and a pressure of from about 13.8 psia to about 80.8 psia. The azeotrope or azeotrope-like composition may consist essentially of from about 88.3 wt. % to about 90.6 wt. % of 1,1,2-trifluoroethane (HFC-143) and from about 11.7 wt. % to about 9.4 wt. % of hydrogen fluoride (HF) at a temperature of from about −0.2° C. to about 49.9° C. and a pressure of from about 13.8 psia to about 80.8 psia. The azeotrope or azeotrope-like composition may consist of from about 88.3 wt. % to about 90.6 wt. % of 1,1,2-trifluoroethane (HFC-143) and from about 11.7 wt. % to about 9.4 wt. % of hydrogen fluoride (HF) at a temperature of from about −0.2° C. to about 49.9° C. and a pressure of from about 13.8 psia to about 80.8 psia.

The homogenous azeotrope or azeotrope-like compositions may comprise from about 83.6 wt. % to about 99.9 wt. % of 1,1,2-trifluoroethane (HFC-143) and from about 0.1 wt. % to about 16.4 wt. % of hydrogen fluoride (HF), and more specifically, from about 86.1 wt. % to about 90.5 wt. % of 1,1,2-trifluoroethane (HFC-143) and from about 9.5 wt. % to about 13.9 wt. % of hydrogen fluoride (HF), and more particularly, from about 87.2 wt. % to about 89.4 wt. % of 1,1,2-trifluoroethane (HFC-143) and from about 10.6 wt. % to about 12.8 wt. % of hydrogen fluoride (HF), and still more specifically, about 88.3 wt. % of 1,1,2-trifluoroethane (HFC-143) and about 11.7 wt. % of hydrogen fluoride (HF) at a pressure of about 13.8 psia.

The homogenous azeotrope or azeotrope-like compositions may consist essentially of from about 83.6 wt. % to about 99.9 wt. % of 1,1,2-trifluoroethane (HFC-143) and from about 0.1 wt. % to about 16.4 wt. % of hydrogen fluoride (HF), and more specifically, from about 86.1 wt. % to about 90.5 wt. % of 1,1,2-trifluoroethane (HFC-143) and from about 9.5 wt. % to about 13.9 wt. % of hydrogen fluoride (HF), and more particularly, from about 87.2 wt. % to about 89.4 wt. % of 1,1,2-trifluoroethane (HFC-143) and from about 10.6 wt. % to about 12.8 wt. % of hydrogen fluoride (HF), and still more specifically, about 88.3 wt. % of 1,1,2-trifluoroethane (HFC-143) and about 11.7 wt. % of hydrogen fluoride (HF) at a pressure of about 13.8 psia.

The homogenous azeotrope or azeotrope-like compositions may consist of from about 83.6 wt. % to about 99.9 wt. % of 1,1,2-trifluoroethane (HFC-143) and from about 0.1 wt. % to about 16.4 wt. % of hydrogen fluoride (HF), and more specifically, from about 86.1 wt. % to about 90.5 wt. % of 1,1,2-trifluoroethane (HFC-143) and from about 9.5 wt. % to about 13.9 wt. % of hydrogen fluoride (HF), and more particularly, from about 87.2 wt. % to about 89.4 wt. % of 1,1,2-trifluoroethane (HFC-143) and from about 10.6 wt. % to about 12.8 wt. % of hydrogen fluoride (HF), and still more specifically, about 88.3 wt. % of 1,1,2-trifluoroethane (HFC-143) and about 11.7 wt. % of hydrogen fluoride (HF) at a pressure of about 13.8 psia.

In other words, the compositions may comprise hydrogen fluoride (HF) in an amount of as much as about 16.4 wt. %, or about 13.9 wt. %, or about 12.8 wt. %, or about 11.7 wt. %, or as little as about 0.1 wt. %, or about 9.5 wt. %, or about 10.6 wt. %, or by any two of the foregoing values as endpoints, such as from about 0.1 wt. % to about 16.4 wt. %, from about 9.5 wt. % to about 13.9 wt. %, from about 10.6 wt. % to about 12.8 wt. %, and/or about 11.7 wt. %, based on the total weight of the 1,1,2-trifluoroethane (HFC-143) and hydrogen fluoride (HF) of the azeotrope or azeotrope-like composition at a pressure of about 13.8 psia.

In other words, the compositions may consist essentially of hydrogen fluoride (HF) in an amount of as much as about 16.4 wt. %, or about 13.9 wt. %, or about 12.8 wt. %, or about 11.7 wt. %, or as little as about 0.1 wt. %, or about 9.5 wt. %, or about 10.6 wt. %, or by any two of the foregoing values as endpoints, such as from about 0.1 wt. % to about 16.4 wt. %, from about 9.5 wt. % to about 13.9 wt. %, from about 10.6 wt. % to about 12.8 wt. %, and/or about 11.7 wt. %, based on the total weight of the 1,1,2-trifluoroethane (HFC-143) and hydrogen fluoride (HF) of the azeotrope or azeotrope-like composition at a pressure of about 13.8 psia.

In other words, the compositions may consist of hydrogen fluoride (HF) in an amount of as much as about 16.4 wt. %, or about 13.9 wt. %, or about 12.8 wt. %, or about 11.7 wt. %, or as little as about 0.1 wt. %, or about 9.5 wt. %, or about 10.6 wt. %, or by any two of the foregoing values as endpoints, such as from about 0.1 wt. % to about 16.4 wt. %, from about 9.5 wt. % to about 13.9 wt. %, from about 10.6 wt. % to about 12.8 wt. %, and/or about 11.7 wt. %, based on the total weight of the 1,1,2-trifluoroethane (HFC-143) and hydrogen fluoride (HF) of the azeotrope or azeotrope-like composition at a pressure of about 13.8 psia.

In other words, the compositions may comprise 1,1,2-trifluoroethane (HFC-143) in an amount as much as about 99.9 wt. %, or about 90.5 wt. %, or about 89.4 wt. %, or about 88.3 wt. %, or as little as about 83.6 wt. %, or about 86.1 wt. %, or about 87.2 wt. %, or by any two of the foregoing values as endpoints, such as from about 83.6 wt. % to about 99.9 wt. %, from about 86.1 wt. % to about 90.5 wt. %, from about 87.2 wt. % to about 89.4 wt. %, and/or about 88.3 wt. %, based on the total weight of the 1,1,2-trifluoroethane (HFC-143) and hydrogen fluoride (HF) of the azeotrope or azeotrope-like composition at a pressure of about 13.8 psia.

In other words, the compositions may consist essentially of 1,1,2-trifluoroethane (HFC-143) in an amount as much as about 99.9 wt. %, or about 90.5 wt. %, or about 89.4 wt. %, or about 88.3 wt. %, or as little as about 83.6 wt. %, or about 86.1 wt. %, or about 87.2 wt. %, or by any two of the foregoing values as endpoints, such as from about 83.6 wt. % to about 99.9 wt. %, from about 86.1 wt. % to about 90.5 wt. %, from about 87.2 wt. % to about 89.4 wt. %, and/or about 88.3 wt. %, based on the total weight of the 1,1,2-trifluoroethane (HFC-143) and hydrogen fluoride (HF) of the azeotrope or azeotrope-like composition at a pressure of about 13.8 psia.

In other words, the compositions may consist of 1,1,2-trifluoroethane (HFC-143) in an amount as much as about 99.9 wt. %, or about 90.5 wt. %, or about 89.4 wt. %, or about 88.3 wt. %, or as little as about 83.6 wt. %, or about 86.1 wt. %, or about 87.2 wt. %, or by any two of the foregoing values as endpoints, such as from about 83.6 wt. % to about 99.9 wt. %, from about 86.1 wt. % to about 90.5 wt. %, from about 87.2 wt. % to about 89.4 wt. %, and/or about 88.3 wt. %, based on the total weight of the 1,1,2-trifluoroethane (HFC-143) and hydrogen fluoride (HF) of the azeotrope or azeotrope-like composition at a pressure of about 13.8 psia.

The compositions may have azeotropic or azeotrope-like characteristics at a temperature of about −0.2° C., about 24.7° C., and/or about 49.9° C., or within any range encompassed by any two of the foregoing values as endpoints, such as from about −0.2° C. to about 49.9° C.

The compositions may have azeotropic or azeotrope-like characteristics at a pressure of about 13.8 psia, about 36.1 psia, about 80.8 psia, or within any range encompassed by any two of the foregoing values as endpoints, such as from about 13.8 psia to about 80.8 psia.

Specifically, and as described in Table 1 below, the azeotrope or azeotrope-like composition of 1,1,2-trifluoroethane (HFC-143) and hydrogen fluoride (HF) may correlate with pressure (psia) and saturation temperature.

In column (i) of Table 1 below, temperature glide is the difference between the saturated vapor temperature and the saturated liquid temperature at a fixed pressure in thermodynamic equilibrium. Consequently, an azeotrope composition may have a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This method was used to determine the relative compositions in column (i) of Table 1 below, which may be regarded as the broadest azeotrope-like composition range.

In column (ii) of Table 1 below, the relative volatility is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This method was used to determine the relative compositions in column (ii) of Table 1 below, which may be regarded as an intermediate azeotrope-like composition range.

In column (iii) of Table 1 below, a relative volatility of 1.05 is substantially close to 1.0 and therefore, compositions that satisfy such relative volatility are considered azeotrope-like. This may be regarded as the narrowest azeotrope-like composition range.

Column (1) of Table 1 below describes the azeotrope composition that, at a given pressure, the azeotrope composition boils at a constant characteristic temperature less than the lower boiling point component (minimum boiling azeotrope). At this characteristic temperature the same composition will exist in both the vapor and liquid phases. Therefore, the components of the azeotrope composition cannot be separated during a phase change and are regarded as the azeotrope composition. Such compositional values are presented in Column (1) of Table 1 below corresponding to each pressure and saturation temperature. In other words, for each row of Table 1, the azeotrope or azeotrope-like composition will exist at that given row's temperature and pressure, and at any compositional wt. % or within any compositional weight percent range wt. % range as defined in any one of columns (1), (i), (ii) and (iii) for the respective row.

Accordingly, and in view of the foregoing, the azeotrope or azeotrope-like composition of 1,1,2-trifluoroethane (HFC-143) and hydrogen fluoride (HF) may comprise any of the values as described in each of columns (1), (i), (ii), or (iii) in each row of Table 1 below.

In another embodiment, the azeotrope or azeotrope-like composition of 1,1,2-trifluoroethane (HFC-143) and hydrogen fluoride (HF) may consist essentially of any of the values as described in each of columns (1), (i), (ii), or (iii) in each row of Table 1 below.

In a further embodiment, the azeotrope or azeotrope-like composition of 1,1,2-trifluoroethane (HFC-143) and hydrogen fluoride (HF) may consist of any of the values as described in each of columns (1), (i), (ii), or (iii) in each row of Table 1 below.

TABLE 1
1) Azeotrope Composition and (2) Azeotrope-like composition ranges
of 1,1,2-trifluoroethane (HFC-143) and hydrogen fluoride (HF)

| | | | | (2) Azeotropic-Like Composition Range | | | | | |
|---|---|---|---|---|---|---|---|---|---|
| | | (1) Azeotropic | | (i) | | (ii) | | (iii) | |
| | | Composition | | Glide <0.5° C. | | Rel. Vol <1.1 | | Rel. Vol <1.05 | |
| | Saturation | Wt. % | | Wt. % | | Wt. % | | Wt. % | |
| Pressure | Temp. | HFC- | Wt. % | HFC- | Wt. % | HFC- | Wt. % | HFC- | Wt. % |
| (psia) | (° C.) | 143 | HF | 143 | HF | 143 | HF | 143 | HF |
| 13.8 | −0.2 | 88.3 | 11.7 | 83.6 to | 0.1 to | 86.1 to | 9.5 to | 87.2 to | 10.6 to |
| | | | | 99.9 | 16.4 | 90.5 | 13.9 | 89.4 | 12.8 |
| 36.1 | 24.7 | 90.1 | 9.9 | 85.5 to | 0.1 to | 87.9 to | 7.8 to | 89.0 to | 8.8 to |
| | | | | 99.9 | 14.5 | 92.2 | 12.1 | 91.2 | 11.0 |
| 80.8 | 49.9 | 90.6 | 9.4 | 86.3 to | 0.1 to | 88.4 to | 7.2 to | 89.5 to | 8.3 to |
| | | | | 99.9 | 13.7 | 92.8 | 11.6 | 91.7 | 10.5 |

### III. Production of E-1,2-Difluoroethylene (HFO-1 132E)

It has been found that azeotrope or azeotrope-like compositions of 1,1,2-trifluoroethane (HFC-143) and hydrogen fluoride (HF) may be formed or otherwise encountered during production of E-1,2-difluoroethylene (HFO-1 132E).

In particular, azeotrope or azeotrope-like compositions of 1,1,2-trifluoroethane (HFC-143) and hydrogen fluoride (HF) may be formed or otherwise encountered in a method for producing E-1,2-difluoroethylene (HFO-1 132E) from a precursor molecule containing an alkene functional group, and particularly, an ethylene group (herein referred to as an “ethylene-based precursor molecule”) of the following formula:

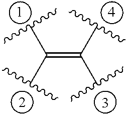

where, R₁, R₂, R₃, and R₄ are selected from any one of H, Cl, F, Br, and I. One nonlimiting example of such a precursor molecule is 1,2-dichloroethylene, in either isomeric configuration (e.g., trans-1,2-dichloroethylene and cis-1,2-dichloroethylene).

Scheme 1 below illustrates an exemplary method of producing E-1,2-difluoroethylene (HFO-1132E) according to a three-step scheme or process. Scheme 1 includes the following three steps: (i) hydrogenating the ethylene-based precursor molecule to produce 1,1,2-trifluoroethane (HFC-143), (ii) dehydrofluorinating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E).

Schematic equations for the three steps of Scheme 1 are represented below:

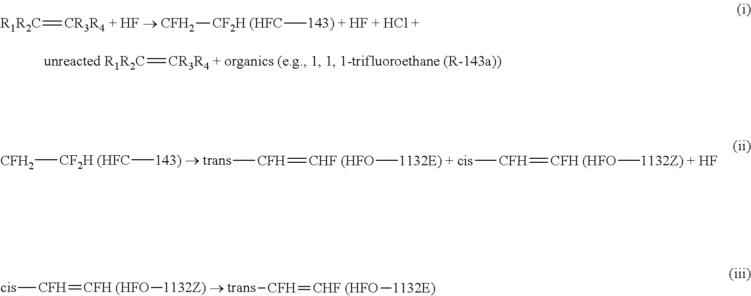

Step (i) may proceed through an intermediate of 1,1,2-trifluoroethene, wherein an ethylene-based precursor molecule is first hydrogenated to produce the 1,1,2-trifluoroethene as an intermediate, which intermediate is itself then hydrogenated to produce 1,1,2-trifluoroethane (HFC-143).

The azeotrope or azeotrope-like compositions of 1,1,2-trifluoroethane (HFC-143) and hydrogen fluoride (HF) are formed in step (i) of Scheme 1 above. Here, the hydrogen fluoride (HF) may itself be a reaction product, where separation of, and subsequent recycling of the hydrogen fluoride (HF) back into the hydrogenation process of Step (i) may enhance the overall recovery of the 1,1,2-trifluoroethane (HFC-143) product. Therefore, it may be important to use or exploit such azeotrope or azeotrope-like compositions of 1,1,2-trifluoroethane (HFC-143) and hydrogen fluoride (HF) to enhance the operation of Scheme 1 to produce E-1,2-difluoroethylene (HFO-1132E) in desired amounts or purities. For example, by separating the azeotrope or azeotrope-like compositions of 1,1,2-trifluoroethane (HFC-143) and hydrogen fluoride (HF), the recovery purity of 1,1,2-trifluoroethane (HFC-143) is enhanced by substantially eliminating the hydrogen fluoride (HF) from the 1,1,2-trifluoroethane (HFC-143). Additionally, since hydrogen fluoride (HF) is itself a reactant in Step (i) of Scheme 1, hydrogen fluoride (HF) may be recycled back into the reaction of Step (i), therefore lowering the amount of makeup hydrogen fluoride (HF) required by Step (i).

The present disclosure additionally relates to a method for producing 1,1,2-trifluoroethane (HFC-143) comprising: hydrogenating an ethylene-based precursor molecule including an alkene and at least one halogen with hydrogen fluoride (HF) to form a product mixture, the product mixture comprising an azeotrope or azeotrope-like composition consisting essentially of effective amounts of 1,1,2-trifluoroethane (HFC-143) and hydrogen fluoride (HF); and separating the 1,1,2-trifluoroethane (HFC-143) and hydrogen fluoride (HF) to provide a product composition comprising the 1,1,2-trifluoroethane (HFC-143). The azeotrope or azeotrope-like composition provided in this method consists essentially of effective amounts of 1,1,2-trifluoroethane (HFC-143) and hydrogen fluoride (HF) as defined in the disclosure above.

The method may further comprise recycling the hydrogen fluoride (HF) to the hydrogenating step. The separating step may comprise conveying the product mixture to an absorption/extractive distillation process. Specifically, water absorption and extractive distillation is described in further detail in Section IV, and sulfuric acid absorption and extractive distillation is described in further detail in Section V, both provided herein.

It will be appreciated that step (i) of Scheme 1 as disclosed above in relation to the production of HFO-1132E discloses a method of producing HFC-143. Therefore, all features disclosed in relation to step (i) of Scheme 1 below, also apply to the method of producing HFC-143.

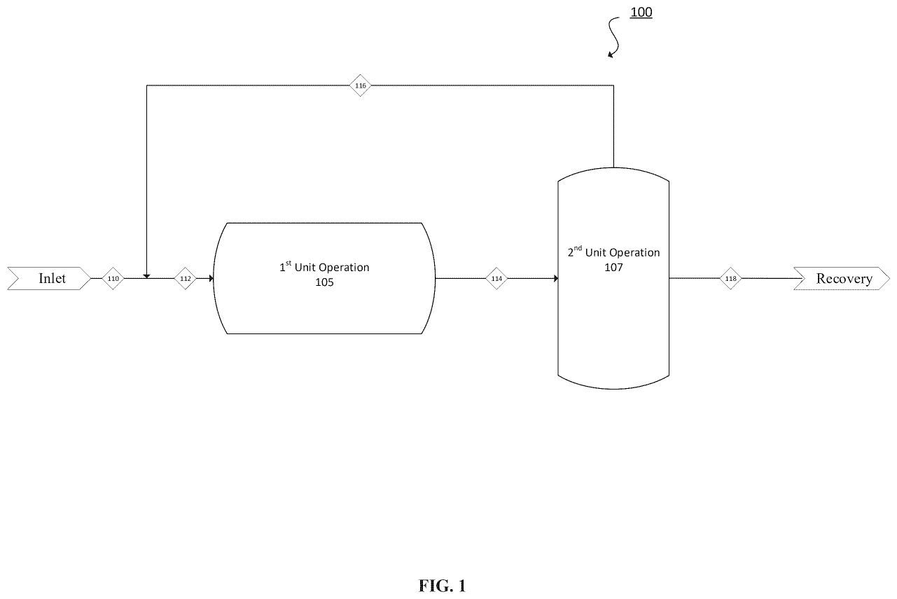

For example, and as illustrated in FIG. 1, an inlet stream 110 to process 100 comprising at least the reactants of Step (i): an ethylene-based precursor molecule including an alkene and at least one halogen+HF, may be mixed with recycle stream 116. Which will be described in further detail herein, forming reactant stream 112. After mixing, reactant stream 112 may be sent to first unit operation 105.

First unit operation 105 may be a hydrogenation reactor whereas Step (i) of Scheme 1 is performed to produce 1,1,2-trifluoroethane (HFC-143). Here, first unit operation 105 may be a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers. The hydrogenation reaction of Step (i) may be carried out in the gas or vapor phase, where the reactor may be first cleaned and flushed with an inert gas such as nitrogen, followed by packing with a catalyst. The catalyst may comprise a metal such as palladium, platinum, rhodium, ruthenium, iron, cobalt, nickel, or antimony. More particularly, the catalyst may be an antimony-based catalyst. The catalyst may be supported on a suitable support, such as carbon or alumina. For instance, the catalyst may be palladium on a carbon support, may be platinum on a carbon support, and/or may be palladium or platinum on an alumina support.

Reactant stream 112 flows through a bed of the catalyst (e.g., in either the up or down direction) within first unit operation 105, undergoing the hydrogenation reaction of Step (i) of Scheme 1. Here, the reaction temperature may be as low as about 100° C., about 125° C., about 150° C., about 200° C., about 250° C. or as high as about 300° C., about 350° C., about 400° C., or within any range encompassed by two of the foregoing values as endpoints, such as from about 100° C. to about 250° C., or from about 150° C. to about 200° C., for example. The temperature may be preferably from about 100° C. to about 350° C., and more preferably from about 200° C. to about 300° C. The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be preferably from about 1 second to about 120 seconds. The pressure may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 300 psig.

Once reacted, reactant stream 112 forms product stream 114 which includes any one of, or combination of HCl, unreacted ethylene-based precursor molecule, 1,1,2-trifluoroethane (HFC-143), unreacted hydrogen fluoride, organics (e.g., 1,1,1-trifluoroethane (R-143a)), and the azeotrope or azeotrope-like composition of and 1,1,2-trifluoroethane (HFC-143) and hydrogen fluoride (HF). As described previously, hydrogen fluoride (HF) is itself a reactant in Step (i). Therefore, the separation of hydrogen fluoride (HF) from 1,1,2-trifluoroethane (HFC-143), and subsequent recycling of hydrogen fluoride (HF) into the reactant stream 112 may be desirable to enhance the recovery of 1,1,2-trifluoroethane (HFC-143), leading to enhanced production of E-1,2-difluoroethylene (HFO-1132E).

Specifically, once exiting first unit operation 105, product stream 114, which includes the azeotrope or azeotrope-like composition of 1,1,2-trifluoroethane (HFC-143) and hydrogen fluoride (HF) may enter second unit operation 107. Second unit operation 107 may separate the components of the azeotrope or azeotrope-like compositions of 1,1,2-trifluoroethane (HFC-143) and hydrogen fluoride (HF) from one another, such as by a combined absorption and extractive distillation process. Specifically, water absorption and extractive distillation is described in further detail in Section IV, and sulfuric acid absorption and extractive distillation is described in further detail in Section V, both provided herein.

Once separated, the 1,1,2-trifluoroethane (HFC-143) is recovered in recovery stream 118, and the reactant hydrogen fluoride (HF) is recycled as recycle stream 116 back into process 100. Here, the amount or purity of 1,1,2-trifluoroethane (HFC-143) in recovery stream 118 may be greater than 90 mol %, 95 mol %, 97 mol %, greater than 99 mol %, or greater than 99.5 mol %, for example, based on total moles of organic components in the composition. The amount of 1,1,2-trifluoroethane (HFC-143) in recovery stream 118 may be less than 5000 ppm, 3000 ppm, 2000 ppm, less than 1000 ppm, less than 500 ppm, or less than 250 ppm, for example, based on total moles of organic components in the composition.

Although unillustrated, recovery stream 118 may be subsequently sent to a further unit operation to separate the HCl from the 1,1,2-trifluoroethane (HFC-143), where the unit operation may be any one of, or combination of, an absorption unit, an adsorption unit, a membrane separator, a cryogenic separator, a subsequent chemical reactor, a distillation unit, and the like. Once the HCl is removed from the 1,1,2-trifluoroethane (HFC-143), the 1,1,2-trifluoroethane (HFC-143) may be utilized as the reactant in Step (ii).

### IV. Separating the Azeotrope and Azeotrope-Like Compositions of 1,1,2-Trifluoroethane (HFC-143) and Hydrogen Fluoride (HF) Via Water Absorption/Extractive Distillation

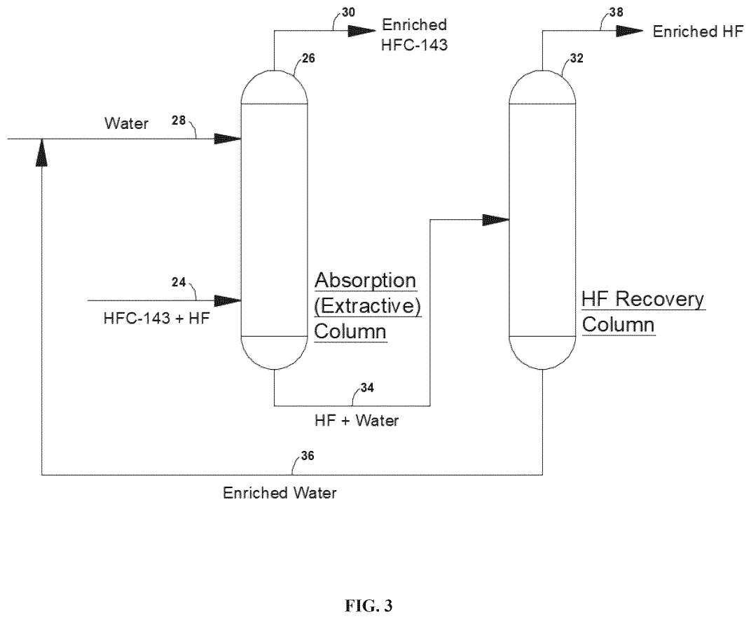

The present disclosure provides a method for separating an azeotrope or azeotrope-like composition of 1,1,2-trifluoroethane (HFC-143) and hydrogen fluoride (HF) via water absorption and/or extractive distillation. Example 5 discusses one such embodiment. A schematic of an exemplary separation apparatus is provided in FIG. 3.

For instance, the absorption method may include conveying a product mixture and water to a first column. The absorption method may further include collecting an overhead product from the first column comprising a first component of the azeotrope or azeotrope-like composition in the form of a gas or liquid. The absorption method may further include collecting a first bottoms product from the first column comprising a mixture of water and a second component of the azeotrope or azeotrope-like composition. The absorption method may further include conveying the first bottoms product to a second column to separate the water and the second component of the azeotrope or azeotrope-like composition. The absorption method may further include removing a composition consisting essentially of the second component of the azeotrope or azeotrope-like composition as a second distillate from the second column.

For instance, the extractive distillation method may include conveying a product mixture and water to a first column. The extractive distillation method may further include collecting a first distillate from the first column comprising a first component of the azeotrope or azeotrope-like composition. The extractive distillation method may further include collecting a first bottoms product from the first column comprising a mixture of the water and a second component of the azeotrope or azeotrope-like composition. The extractive distillation method may further include conveying the first bottoms product to a second column to separate the water and the second component of the azeotrope or azeotrope-like composition. The extractive distillation method may further include removing a composition consisting essentially of the second component of the azeotrope or azeotrope-like composition as a second distillate from the second column.

A schematic of an exemplary separation apparatus is provided in FIG. 3. Referring to this figure, product stream 24 (which may be the same as product stream 114 with reference to FIG. 1), which includes at least the azeotrope or azeotrope-like composition of 1,1,2-trifluoroethane (HFC-143) and hydrogen fluoride (HF), is conveyed to absorption/extractive column 26 along with water 28. Here, hydrogen fluoride (HF) found within the azeotrope or azeotrope-like composition of the product stream 24, dissolves in the water 28 more readily than 1,1,2-trifluoroethane (HFC-143). The water 28 therefore may be used to “break” the azeotrope or azeotrope-like composition into individual components, based upon the affinity/solubility difference, and therefore, utilized to selectively separate each of the components of the azeotrope or azeotrope-like composition. For example, the water 28 has a higher affinity for hydrogen fluoride (HF) and may act as a selective absorber/solvent for the hydrogen fluoride (HF) of the azeotrope or azeotrope-like composition.

Absorption/Extractive column 26 is operated such that the operational parameters (e.g. temperature and pressure) separate the mixture of the water 28 and the first/dissolved component of the azeotrope or azeotrope-like composition from the second component of the azeotrope or azeotrope-like composition. For example, the water 28 has a higher affinity for hydrogen fluoride (HF) and therefore water 28 and hydrogen fluoride (HF) are recovered in bottoms product 34 from absorption/extractive column 26, while enriched 1,1,2-trifluoroethane (HFC-143) is recovered as the overhead product/distillate 30.

In a second step, the bottoms product 34 containing each of the mixture of the water and the dissolved first component of the azeotrope or azeotrope-like composition, namely hydrogen fluoride (HF), is conveyed to recovery column 32. Recovery column 32 is operated such that the operational parameters (e.g. temperature and pressure) separate the water and hydrogen fluoride (HF) from one another, where the enriched water is recovered as bottoms product 36 and the enriched hydrogen fluoride (HF) is recovered as in the distillate 38. The recovered enriched water 36 may be recycled and used as water 28 in the absorption/extractive column 26, as described previously.

### V. Separating Azeotrope and Azeotrope-Like Compositions of 1,1,2-Trifluoroethane (HFC-143) and Hydrogen Fluoride (HF) Via Sulfuric Acid Absorption/Extractive Distillation

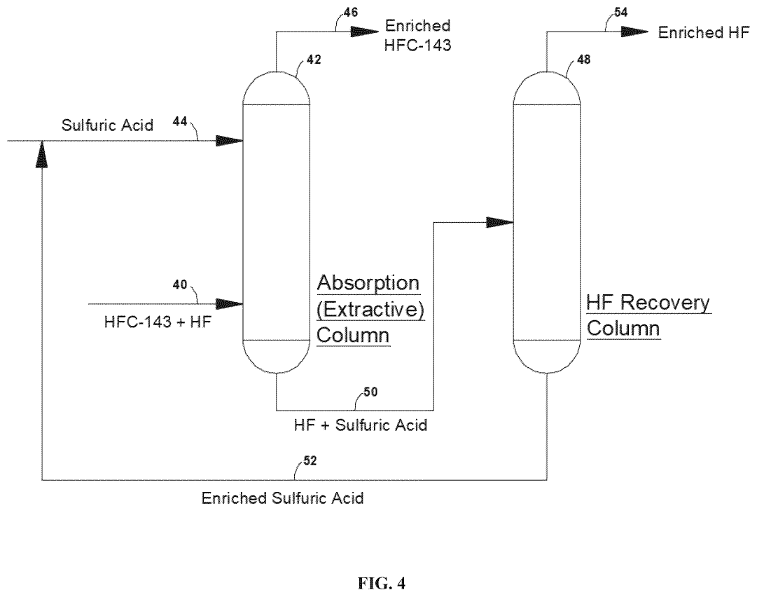

The present disclosure provides a method for separating an azeotrope or azeotrope-like composition of 1,1,2-trifluoroethane (HFC-143) and hydrogen fluoride (HF) via sulfuric acid (H₂SO₄) absorption and/or extractive distillation. Example 6 discusses one such embodiment. A schematic of an exemplary separation apparatus is provided in FIG. 4.

For instance, the absorption method may include conveying a product mixture and sulfuric acid (H₂SO₄) to a first column. The absorption method may further include collecting an overhead product from the first column comprising a first component of the azeotrope or azeotrope-like composition in the form of a gas or liquid. The absorption method may further include collecting a first bottoms product from the first column comprising a mixture of sulfuric acid (H₂SO₄) and a second component of the azeotrope or azeotrope-like composition. The absorption method may further include conveying the first bottoms product to a second column to separate the sulfuric acid (H₂SO₄) and the second component of the azeotrope or azeotrope-like composition. The absorption method may further include removing a composition consisting essentially of the second component of the azeotrope or azeotrope-like composition as a second distillate from the second column.

For instance, the extractive distillation method may include conveying a product mixture and sulfuric acid (H₂SO₄) to a first column. The extractive distillation method may further include collecting a first distillate from the first column comprising a first component of the azeotrope or azeotrope-like composition. The extractive distillation method may further include collecting a first bottoms product from the first column comprising a mixture of the sulfuric acid (H₂SO₄) and a second component of the azeotrope or azeotrope-like composition. The extractive distillation method may further include conveying the first bottoms product to a second column to separate the sulfuric acid (H₂SO₄) and the second component of the azeotrope or azeotrope-like composition. The extractive distillation method may further include removing a composition consisting essentially of the second component of the azeotrope or azeotrope-like composition as a second distillate from the second column.

A schematic of an exemplary separation apparatus is provided in FIG. 4. Referring to this figure, product stream 40 (which may be the same as product stream 114 with reference to FIG. 1), which includes at least the azeotrope or azeotrope-like composition of 1,1,2-trifluoroethane (HFC-143) and hydrogen fluoride (HF), is conveyed to absorption/extractive column 42 along with sulfuric acid (H₂SO₄) 44. Here, hydrogen fluoride (HF) found within the azeotrope or azeotrope-like composition of the product stream 40, dissolves in the sulfuric acid (H₂SO₄) 44 more readily than 1,1,2-trifluoroethane (HFC-143). The sulfuric acid (H₂SO₄) 44 therefore may be used to “break” the azeotrope or azeotrope-like composition into individual components, based upon the affinity/solubility difference, and therefore, utilized to selectively separate each of the components of the azeotrope or azeotrope-like composition. For example, the sulfuric acid (H₂SO₄) 44 has a higher affinity for hydrogen fluoride (HF) and may act as a selective absorber/solvent for the hydrogen fluoride (HF) of the azeotrope or azeotrope-like composition.

Absorption/Extractive column 42 is operated such that the operational parameters (e.g. temperature and pressure) separate the mixture of the sulfuric acid (H₂SO₄) 44 and the first/dissolved component of the azeotrope or azeotrope-like composition from the second component of the azeotrope or azeotrope-like composition. For example, the sulfuric acid (H₂SO₄) 44 has a higher affinity for hydrogen fluoride (HF) and therefore sulfuric acid (H₂SO₄) 44 and hydrogen fluoride (HF) are recovered in bottoms product 50 from absorption/extractive column 42, while enriched 1,1,2-trifluoroethane (HFC-143) is recovered as the overhead product/distillate 46.

In a second step, the bottoms product 50 containing each of the mixture of the sulfuric acid (H₂SO₄) and the dissolved first component of the azeotrope or azeotrope-like composition, namely hydrogen fluoride (HF), is conveyed to recovery column 48. Recovery column 48 is operated such that the operational parameters (e.g. temperature and pressure) separate the sulfuric acid (H₂SO₄) and hydrogen fluoride (HF) from one another, where the enriched sulfuric acid (H₂SO₄) is recovered as bottoms product 52 and the enriched hydrogen fluoride (HF) is recovered as in the distillate 54. The recovered enriched sulfuric acid (H₂SO₄) 52 may be recycled and used as sulfuric acid (H₂SO₄) 44 in the absorption/extractive column 42, as described previously.

### Examples

### Example 1: Hydrofluorination of an Ethylene-Based Precursor Molecule:Trans-1,2-Dichloroethylene, to Form 1,1,2-Trifluoroethane (HFC-143)

An agitated Teflon lined 8″ ID X 11″ L reactor with a L/D ratio of ˜1.4/1 is used to conduct the hydrofluorination reaction of the ethylene-based precursor molecule:trans-1,2-dichloroethylene. The reactor has a SiC dip tube to allow HF and organic feeds to bubble through the liquid Sb catalyst, a multiple point temperature probe to measure reaction temperature, and a vapor port for charging catalyst and releasing reactor pressure when needed. The reaction system consists of the agitated reactor, a catalyst stripper which is connected to the reactor, feed (organic, HF, and C12) delivery systems, acid scrubber, drier, and product collection system. The reaction effluent from the catalyst stripper is directed to a to KOH scrubber for HF/HCl removal. The acid-free product is then sent to a drying column for moisture removal before being condensed in a PCC (Product Collection Cylinder).

After the reactor is pressure tested and purged with nitrogen, 1 kg of SbCl₅ catalyst is charged into reactor. After the reactor is pressure checked again, fluorination of catalyst is initiated by introducing HF into reactor. After initial exotherm, reactor is heated up to target temperatures (85-90° C.) and target pressure (100 psig) while HF flow is continued. After 7 lbs of HF is added, HF flow is adjected to target rate (0.5-0.7 lb/h), and trans-1,2-dichloroethylene feed (0.6-0.8 lb/h) is started with an HF/trans-1,2-dichloroethylene molar ratio ranged from 3/1 to 6/1. A dririte column is used to remove any moisture included in organic feed. During reaction, effort is made to maintain relatively constant reactor weight and samples are taken at the outlet of catalyst stripper for the analyses of organic and acid contents. The analysis results indicate the reaction effluent from the catalyst stripper comprises 1,1,2-trifluoroethane (HFC-143), hydrogen fluroide (HF), HCl, trans-1,2-dichloroethylene, and other organic components such as 1,1,1-trifluoroethane.

### Example 2: Hydrofluorination of an Ethylene-Based Precursor Molecule:Cis-1,2-Dichloroethylene, to Form 1,1,2-Trifluoroethane

An agitated Teflon lined 8″ ID X 11″ L reactor with a L/D ratio of 1.4/1 is used to conduct the hydrofluorination reaction of the ethylene-based precursor molecule:cis-1,2-dichloroethylene. The reactor has a SiC dip tube to allow HF and organic feeds to bubble through the liquid Sb catalyst, a multiple point temperature probe to measure reaction temperature, and a vapor port for charging catalyst and releasing reactor pressure when needed. The reaction system consists of the agitated reactor, a catalyst stripper which is connected to the reactor, feed (organic, HF, and C12) delivery systems, acid scrubber, drier, and product collection system. The reaction effluent from the catalyst stripper is directed to a to KOH scrubber for HF/HCl removal. The acid-free product is then sent to a drying column for moisture removal before being condensed in a PCC (Product Collection Cylinder).

After the reactor is pressure tested and purged with nitroge, 1 kg of SbCl₅ catalyst is charged into reactor. After the reactor is pressure checked again, fluorination of catalyst is initiated by introducing HF into reactor. After initial exotherm, reactor is heated up to target temperatures (85-90° C.) and target pressure (100 psig) while HF flow is continued. After 7 lbs of HF is added, HF flow is adjected to target rate (0.5-0.7 lb/h), and cis-1,2-dichloroethylene feed (0.6-0.8 lb/h) is started with an HF/cis-1,2-dichloroethylene molar ratio ranged from 3/1 to 6/1. A dririte column is used to remove any moisture included in organic feed. During reaction, effort is made to maintain relatively constant reactor weight and samples are taken at the outlet of catalyst stripper for the analyses of organic and acid contents. The analysis results indicate the reaction effluent from the catalyst stripper comprises 1,1,2-trifluoroethane (HFC-143), hydrogen fluoride (HF), HCl, cis-1,2-dichloroethylene, and other organic components such as 1,1,1-trifluoroethane.

### Example 3: Measurement and Characterization of Azeotrope and Azeotrope-Like Compositions of 1,1,2-Trifluoroethane (HFC-143) and Hydrogen Fluoride (HF)

A volume calibrated PTx cell was used to measure azeotrope and azeotrope-like compositions of 1,1,2-trifluoroethane (HFC-143) and hydrogen fluoride (HF). Mixtures of HFC-143 and HF were gravimetrically prepared, and in successive increments, into an evacuated PTx cell. Once prepared, the PTx cell was inserted into a thermostatted bath. In the bath, the cell was attached to an instrumentation manifold equipped with calibrated pressure transducers and resistance temperature detectors (RTD); this provided a means to measure and record the total saturation pressure of each cell's contents at its local temperature.

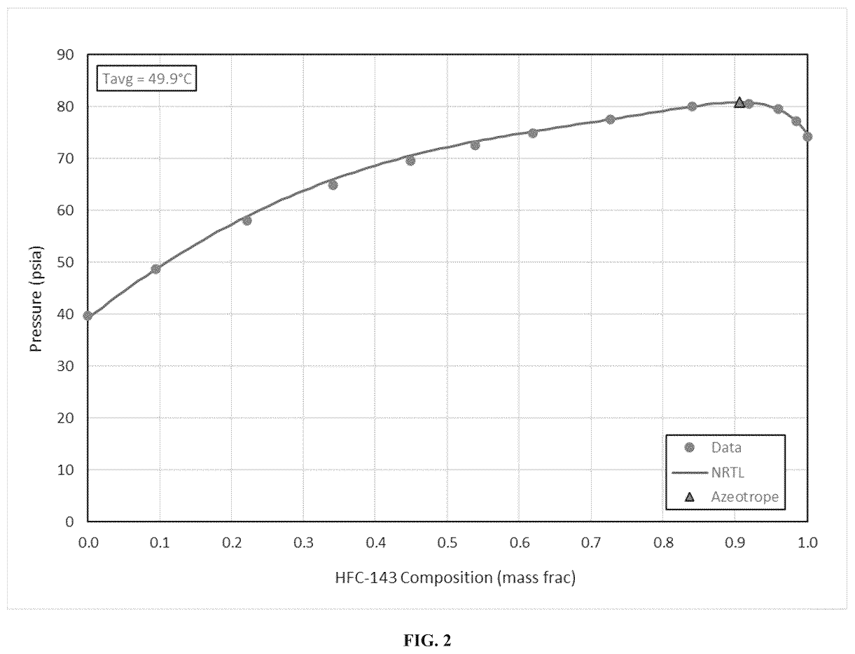

To establish equilibrium at a target temperature, the set point of the bath was adjusted, yielding an average temperature (T_{avg}) of 49.9° C. across all of the PTx cell's compositions. Once at equilibrium, recognized as when temperature and pressure remained stable for several hours, the local temperature and saturation pressures of each cell were recorded. From these pressure-temperature-composition data, the binary interaction parameters of HFC-143 and HF for the Non-random, Two-Liquid (NRTL) activity coefficient model were identified. As indicated by the maximum pressure shown in FIG. 2, a minimum boiling, homogeneous azeotrope composition of about 90.6 wt. % HFC-143 and about 9.4 wt. % HF was formed based on the data presented in Table 2.

TABLE 2
PTx Study of HFC-143 and HF at an average temperature
of 49.9° C. Maximum pressure observed between
84.0 wt. % HFC-143 and 96.0 wt. % HFC-143, azeotrope
formed at about 90.6 wt. % HFC-143.

| HFC-143 | | |
|---|---|---|
| Composition | HF Composition | Pressure |
| (wt. %) | (wt. %) | (psia) |
| 0.0 | 100.0 | 39.71 |
| 9.5 | 90.5 | 48.74 |
| 22.2 | 77.8 | 58.07 |
| 34.1 | 65.9 | 64.84 |
| 44.9 | 55.1 | 69.53 |
| 53.9 | 46.1 | 72.52 |
| 61.9 | 38.1 | 74.92 |
| 72.6 | 27.4 | 77.56 |
| 84.0 | 16.0 | 79.95 |
| 92.0 | 8.0 | 80.57 |
| 96.0 | 4.0 | 79.56 |
| 98.5 | 1.5 | 77.16 |
| 100.0 | 0.0 | 74.13 |

### Example 4: Azeotrope Locus

The procedure of Example 3 was repeated for each of the temperatures indicated in Table 3 below to generate the azeotropic and azeotrope-like compositional ranges.

Table 3 below includes the azeotrope and azeotrope-like compositions for the HFC-143 and HF:

TABLE 3
Azeotrope Locus

| | | | | (2) Azeotropic-Like Composition Range | | | | | |
|---|---|---|---|---|---|---|---|---|---|
| | | (1) Azeotropic | | (i) | | (ii) | | (iii) | |
| | | Composition | | Glide <0.5° C. | | Rel. Vol <1.1 | | Rel. Vol <1.05 | |
| | Saturation | Wt. % | | Wt. % | | Wt. % | | Wt. % | |
| Pressure | Temp. | HFC- | Wt. % | HFC- | Wt. % | HFC- | Wt. % | HFC- | Wt. % |
| (psia) | (° C.) | 143 | HF | 143 | HF | 143 | HF | 143 | HF |
| 13.8 | −0.2 | 88.3 | 11.7 | 83.6 to | 0.1 to | 86.1 to | 9.5 to | 87.2 to | 10.6 to |
| | | | | 99.9 | 16.4 | 90.5 | 13.9 | 89.4 | 12.8 |
| 36.1 | 24.7 | 90.1 | 9.9 | 85.5 to | 0.1 to | 87.9 to | 7.8 to | 89.0 to | 8.8 to |
| | | | | 99.9 | 14.5 | 92.2 | 12.1 | 91.2 | 11.0 |
| 80.8 | 49.9 | 90.6 | 9.4 | 86.3 to | 0.1 to | 88.4 to | 7.2 to | 89.5 to | 8.3 to |
| | | | | 99.9 | 13.7 | 92.8 | 11.6 | 91.7 | 10.5 |

In view of the above data, temperature glide and relative volatility were applied to determine the azeotrope and azeotrope-like compositions.

The temperature glide and relative volatility of a mixture may be derived from thermodynamic measurements, such as those collected via PTx, subject to material balance and thermodynamic constraints. Several methods for deriving temperature glide from thermodynamic measurements are described in Sandier, S. I. (2006). Chapter 10:Vapor-Liquid Equilibrium in Mixtures. In Chemical, Biochemical, and Engineering Thermodynamics (4th ed., pp. 489-574) which includes constraining thermodynamic consistency through the fundamental Gibbs-Duhem relationship and resolving the vapor phase composition, from the measurements, through combined mass balance and equilibrium criteria (frequently referred to as the Rachford-Rice equation or algorithm). Through this derivation, the relationship between equilibrium compositions, temperatures, and pressures are established permitting temperature glide and relative volatility to be evaluated.

For a given composition, the temperature glide, by definition, is the difference between the saturated vapor temperature and the saturated liquid temperature at a fixed pressure in thermodynamic equilibrium. Consequently, an azeotrope composition has a temperature glide of zero and an azeotrope-like composition has a temperature glide that is substantially close to zero. It has been identified that a temperature glide less than 0.5° C. is substantially close to zero and therefore compositions that satisfy such temperature glide are considered azeotrope-like. This is the broad azeotrope-like range.

The relative volatility, by definition, is the ratio of the vapor composition to the liquid composition of the most volatile component relative to the ratio of the vapor composition to the liquid composition of the less volatile component at a fixed pressure in thermodynamic equilibrium. Consequently, an azeotrope composition has a relative volatility of 1.0 and an azeotrope-like composition has a relative volatility that is substantially close to 1.0. It has been identified that a relative volatility of 1.1 is substantially close to 1.0 and therefore compositions that satisfy such relative volatility are considered azeotrope-like. This is the intermediate azeotrope-like range.

Additionally, it has been identified that a relative volatility of 1.05 is substantially close to 1.0 and therefore compositions that satisfy such relative volatility are considered azeotrope-like. This is the narrow azeotrope-like range.

### Example 5: Water Absorption/Extractive Distillation

A well-known consequence of azeotropic mixtures is the inability to fully separate its constituents in distillation operation. For example, separation of a 50/50 mass % mixture 1,1,2-trifluoroethane (HFC-143) and hydrogen fluoride (HF) by a distillation column held at 13.8 psia, exhibiting azeotropic behavior as described by Example 4, would be bounded by compositions between the HF endpoint and the minimum boiling azeotropic composition. In other words, distillation of a mixture at these conditions would be unable to produce HFC-143 in a purity greater than 88.3 mass %. To address this fundamental barrier of azeotropes and attain both purer HFC-143 and HF, a different separation strategy must be realized, such as pressure swing, absorption, and/or extractive distillation.

Initially, a stream containing the azeotrope or azeotrope-like. As noted by Example 4, the azeotropic composition of HFC-143 and HF is sensitive to pressure. This sensitivity can be exploited to support better separation through pressure swing distillation. In this system, a pressure-sensitive azeotrope is separated using two distillation columns in sequence. Specifically, and perhaps in addition to pressure effects, a composition of 1,1,2-trifluoroethane (HFC-143) and hydrogen fluoride (HF) is fed to a first absorption/extractive distillation column, along with water as an entrainer fluid. The absorption/extraction column is operated at a suitable temperature and pressure. The first distillation column acts as an extraction column, where the water entrainer fluid and one of the components of the azeotrope or azeotrope-like composition (e.g., component A: HF) is recovered in the bottoms product from the extraction column, and the other component of the azeotrope or azeotrope-like composition (e.g., component B: HFC-143). Here, the water entrainer fluid is selected based upon various thermodynamic properties which includes an affinity difference between component A to dissolve in water vs. component B, where substantially all of the component A is dissolved in the water, while little to none of the component B is dissolved in the water. The column is operated based upon thermodynamic differences between entrainer/component A and the component B, similar to those described in relation to Section IV above. The extraction column is operated such that distillate comprises substantially all of the component B, and the bottoms product comprises substantially all of the entrainer fluid and dissolved component A.

The recovered water entrainer fluid and dissolved component A is fed to a second recovery column operated at a suitable temperature and pressure. The recovery column separates the water from the component A, as based upon thermodynamic differences. Here, substantially all of the water entrainer fluid is recovered in the bottoms product from the recovery column, and substantially all of the component A is recovered in the distillate. The recovered entrainer fluid is thereafter recycled to the extraction column.

### Example 6: Sulfuric Acid Absorption/Extractive Distillation

An azeotrope or azeotrope-like composition of 1,1,2-trifluoroethane (HFC-143) and hydrogen fluoride (HF) is separated by extractive distillation and absorption. Initially, a stream containing an azeotrope or azeotrope-like composition of 1,1,2-trifluoroethane (HFC-143) and hydrogen fluoride (HF) is fed to a first absorption/extractive distillation column, along with Sulfuric Acid (H₂SO₄) as an entrainer fluid. The absorption/extraction column is operated at a suitable temperature and pressure, such as those described in Example 5 previously. The first distillation column acts as an extraction column, where the Sulfuric acid entrainer fluid and one of the components of the azeotrope or azeotrope-like composition (e.g., component A:HF) is recovered in the bottoms product from the extraction column, and the other component of the azeotrope or azeotrope-like composition (e.g., component B:HFC-143). Here, the sulfuric acid entrainer fluid is selected based upon various thermodynamic properties which includes an affinity difference between component A to dissolve in Sulfuric Acid vs. component B, where substantially all of the component A is dissolved in the H₂SO₄, while little to none of the component B is dissolved in the H₂SO₄. The column is operated based upon thermodynamic differences between entrainer/component A and the component B, similar to those described in relation to Example 5. The extraction column is operated such that distillate comprises substantially all of the component B, and the bottoms product comprises substantially all of the entrainer fluid and dissolved component A.

The recovered sulfuric acid entrainer fluid and dissolved component A is fed to a second recovery column operated at a suitable temperature and pressure. The recovery column separates the water from the component A, as based upon thermodynamic differences. Here, substantially all of the H₂SO₄ entrainer fluid is recovered in the bottoms product from the recovery column, and substantially all of the component A is recovered in the distillate. The recovered entrainer fluid is thereafter recycled to the extraction column.

### Example 7: Process for Producing E-1,2-Difluoroethylene (HFO-1132E) from 1,1,2-Trifluoroethane (HFC-143) Via the Separation of an Azeotrope of 1,1,2-Trifluoroethane (HFC-143) and Hydrogen Fluoride (HF)

A reactant stream including an ethylene-based precursor molecule, such as trans-1,2-dichloroethylene or cis-1,2-dichloroethylene, as well as hydrogen fluoride (HF) is provided to a hydrogenation reactor. The hydrogenation reactor is operated at a temperature between 200° C. to 300° C., at a pressure between 10 psig to 200 psig, and for a contact time between 1 second to 60 seconds. The hydrogenation reactor contains a catalyst comprising antimony. After reacting, the product stream from the reaction is analyzed by the PTx method, similar to Example 2 above.

The product stream is found to contain at least HCl and an azeotrope or azeotrope-like composition of 1,1,2-trifluoroethane (HFC-143) and hydrogen fluoride (HF). Specifically, the azeotrope or azeotrope-like composition of 1,1,2-trifluoroethane (HFC-143) and hydrogen fluoride (HF) is found to contain about 83.6 wt. % to 99.9 wt. % of 1,1,2-trifluoroethane (HFC-143) and from about 0.1 wt. % to 16.4 wt. % of hydrogen fluoride (HF). The product stream is subsequently separated to separate the azeotrope or azeotrope-like composition of 1,1,2-trifluoroethane (HFC-143) and hydrogen fluoride (HF) to individual components, where the 1,1,2-trifluoroethane (HFC-143) and the hydrogen fluoride (HF) are further separated from one another.

For example, the azeotrope or azeotrope-like composition of 1,1,2-trifluoroethane (HFC-143) and hydrogen fluoride (HF) is “broken” and separated by absorption/extractive distillation, as described with reference to Examples 5 and 6 above. Hydrogen fluoride (HF) is removed and recycled during the separation process, and the resulting product stream comprises the 1,1,2-trifluoroethane (HFC-143). The final product recovered is found to contain between 90 mol % and 99.5 mol % of 1,1,2-trifluoroethane (HFC-143). Thereafter, the 1,1,2-trifluoroethane (HFC-143) is reacted through the reaction process of Steps (ii) and (iii) of Scheme 1 above to form E-1,2-difluoroethylene (HFO-1132E).

### Aspects

Aspect 1 is a homogenous azeotrope or azeotrope-like composition consisting essentially of effective amounts of 1,1,2-trifluoroethane (HFC-143) and hydrogen fluoride (HF).
Aspect 2 is the homogenous azeotrope or azeotrope-like composition of Aspect 1, consisting essentially of from about 83.6 wt. % to about 99.9 wt. % of 1,1,2-trifluoroethane (HFC-143) and from about 0.1 to about 16.4 wt. % of hydrogen fluoride (HF).
Aspect 3 is the homogenous azeotrope or azeotrope-like composition of aspect 1, consisting essentially of from about 86.1 wt. % to about 90.5 wt. % of 1,1,2-trifluoroethane (HFC-143) and from about 9.5 wt. % to about 13.9 wt. % of hydrogen fluoride (HF).
Aspect 4 is the homogenous azeotrope or azeotrope-like composition of Aspect 1, consisting essentially of from about 87.2 wt. % to about 89.4 wt. % of 1,1,2-trifluoroethane (HFC-143) and from about 10.6 wt. % to about 12.8 wt. % of hydrogen fluoride (HF).
Aspect 5 is the homogenous azeotrope or azeotrope-like composition of Aspect 1, consisting essentially of about 88.3 wt. % of 1,1,2-trifluoroethane (HFC-143) and about 11.7 wt. % of hydrogen fluoride (HF).
Aspect 6 is the homogenous azeotrope or azeotrope-like composition of any one of aspects 1 through 5, wherein the azeotrope or azeotrope-like composition has a boiling point of from about −0.2° C. at a pressure of about 13.8 psia, to about 49.9° C. at a pressure of about 80.8 psia.
Aspect 7 is a method for producing 1,1,2-trifluoroethane (HFC-143) comprising: hydrogenating an ethylene-based precursor molecule of the formula R₁R₂C=CR₃R₄, wherein R₁, R₂, R₃, and R₄ are selected from H, Cl, F, Br and I, with hydrogen fluoride (HF) to form a product mixture, the product mixture comprising a homogenous azeotrope or azeotrope-like composition consisting essentially of effective amounts of 1,1,2-trifluoroethane (HFC-143) and hydrogen fluoride (HF); and separating the 1,1,2-trifluoroethane (HFC-143) and hydrogen fluoride (HF) to provide a product composition comprising the 1,1,2-trifluoroethane (HFC-143).
Aspect 8 is the method of Aspect 7, further comprising recycling the hydrogen fluoride (HF) from the product mixture to the hydrogenating step.
Aspect 9 is the method of either one of Aspects 7 or 8, wherein the separating step comprises: conveying the product mixture to a first absorption/extractive column; collecting a first product from the first column; and conveying a first extract from the first column to a second recovery column to provide a second extract and a second product from the second column.
Aspect 10 is the method of Aspect 9, wherein the first column is an absorption/extractive column comprising water, the first product consists essentially of 1,1,2-trifluoroethane (HFC-143) and the second product consists essentially of hydrogen fluoride (HF).
Aspect 11 is the method of either one of Aspects 9 or 10, wherein the first column is an absorption/extractive column comprising sulfuric acid, the first product consists essentially of 1,1,2-trifluoroethane (HFC-143) and the second product consists essentially of hydrogen fluoride (HF).
Aspect 12 is the method of claim 7, wherein the separating step comprises: conveying the product mixture and an entrainer fluid to a first column; collecting a first extract from the first column comprising a first component of the azeotrope or azeotrope-like composition; collecting a first product from the first column comprising a second component of the azeotrope or azeotrope-like composition; conveying the first extract to a second column to separate the entrainer and the second component of the azeotrope or azeotrope-like composition; and collecting a second product from the second column comprising the second component of the azeotrope or azeotrope-like composition.
Aspect 13 is the method of Aspect 12, wherein the first component of the azeotrope or azeotrope-like composition consisting essentially of 1,1,2-trifluoroethane (HFC-143), and the second component of the azeotrope or azeotrope-like composition consists of hydrogen fluoride (HF).
Aspect 14 is the method of Aspect 7, further comprising reacting the 1,1,2-trifluoroethane (HFC-143) to form trans-1,2-difluoroethylene (HFO-1132E).

What is claimed is:

1. A homogenous azeotrope or azeotrope-like composition consisting essentially of effective amounts of 1,1,2-trifluoroethane (HFC-143) and hydrogen fluoride (HF).

2. The homogenous azeotrope or azeotrope-like composition of claim 1, consisting essentially of from about 83.6 wt. % to about 99.9 wt. % of 1,1,2-trifluoroethane (HFC-143) and from about 0.1 to about 16.4 wt. % of hydrogen fluoride (HF).

3. The homogenous azeotrope or azeotrope-like composition of claim 1, consisting essentially of from about 86.1 wt. % to about 90.5 wt. % of 1,1,2-trifluoroethane (HFC-143) and from about 9.5 wt. % to about 13.9 wt. % of hydrogen fluoride (HF).

4. The homogenous azeotrope or azeotrope-like composition of claim 1, consisting essentially of from about 87.2 wt. % to about 89.4 wt. % of 1,1,2-trifluoroethane (HFC-143) and from about 10.6 wt. % to about 12.8 wt. % of hydrogen fluoride (HF).

5. The homogenous azeotrope or azeotrope-like composition of claim 1, consisting essentially of about 88.3 wt. % of 1,1,2-trifluoroethane (HFC-143) and about 11.7 wt. % of hydrogen fluoride (HF).

6. The homogenous azeotrope or azeotrope-like composition of claim 1, wherein the azeotrope or azeotrope-like composition has a boiling point of from about −0.2° C. at a pressure of about 13.8 psia, to about 49.9° C. at a pressure of about 80.8 psia.

7. A method for producing 1,1,2-trifluoroethane (HFC-143) comprising:
hydrogenating an ethylene-based precursor molecule of the formula R₁R₂C=CR₃R₄, wherein R₁, R₂, R₃, and R₄ are selected from H, Cl, F, Br and I, with hydrogen fluoride (HF) to form a product mixture, the product mixture comprising a homogenous azeotrope or azeotrope-like composition consisting essentially of effective amounts of 1,1,2-trifluoroethane (HFC-143) and hydrogen fluoride (HF); and
separating the 1,1,2-trifluoroethane (HFC-143) and hydrogen fluoride (HF) to provide a product composition comprising the 1,1,2-trifluoroethane (HFC-143).

8. The method of claim 7, further comprising recycling the hydrogen fluoride (HF) from the product mixture to the hydrogenating step.

9. The method of claim 7, wherein the separating step comprises:
conveying the product mixture to a first absorption/extractive column;
collecting a first product from the first column;
conveying a first extract from the first column to a second recovery column to provide a second extract and a second product from the second column.

10. The method of claim 9, wherein the first column is an absorption/extractive column comprising water, the first product consists essentially of 1,1,2-trifluoroethane (HFC-143) and the second product consists essentially of hydrogen fluoride (HF).

11. The method of claim 9, wherein the first column is an absorption/extractive column comprising sulfuric acid, the first product consists essentially of 1,1,2-trifluoroethane (HFC-143) and the second product consists essentially of hydrogen fluoride (HF).

12. The method of claim 7, wherein the separating step comprises:
conveying the product mixture and an entrainer fluid to a first column;
collecting a first extract from the first column comprising a first component of the azeotrope or azeotrope-like composition;
collecting a first product from the first column comprising a second component of the azeotrope or azeotrope-like composition;
conveying the first extract to a second column to separate the entrainer and the second component of the azeotrope or azeotrope-like composition; and
collecting a second product from the second column comprising the second component of the azeotrope or azeotrope-like composition.

13. The method of claim 12, wherein the first component of the azeotrope or azeotrope-like composition consisting essentially of 1,1,2-trifluoroethane (HFC-143), and the second component of the azeotrope or azeotrope-like composition consists of hydrogen fluoride (HF).

14. The method of claim 7, further comprising reacting the 1,1,2-trifluoroethane (HFC-143) to form trans-1,2-difluoroethylene (HFO-1132E).

15. The method of claim 7, wherein the ethylene-based precursor molecule is selected from trans-1,2-dichloroethylene, cis-1,2-dichloroethylene.
